(19) United States
(12) Patent Application Publication
(10) Pub. No.: US 2025/0342969 A1
(43) Pub. Date: Nov. 6, 2025

(54) DIGITAL PATHOLOGY SYSTEM

(71) Applicant: AGENDIA NV, Amsterdam (NL)

(72) Inventors: Annuska Maria GLAS, Irvine, CA (US); Marc Arnaud HOGENBIRK, Amsterdam (NL); Ebrahim HAZRATI, Amsterdam (NL); Josien Carolien HAAN, Amsterdam (NL)

(21) Appl. No.: 18/864,145

(22) PCT Filed: May 11, 2023
(86) PCT No.: PCT/IB2023/000255

§ 371 Date: Nov. 8, 2024

Related U.S. Application Data
(60) Provisional application No. 63/340,527, filed on May 11, 2022.

(51) Int. Cl. G16H 50/30; G16B 40/00; G16H 10/60; G16H 50/70

(52) U.S. Cl. CPC G16H 50/30; G16B 40/00; G16H 10/60; G16H 50/70

(57) ABSTRACT

The invention provides methods and artificial-intelligence (AI) systems that predict disease risk from samples. In particular, AI systems predict diseases risk from samples such as whole slide images (WSIs). When those AI-predicted risks do not match risk scores calculated in laboratory assays, methods of the invention give the AI system further, additional training on select samples. In particular, the samples for additional training are selected for their representation of biological processes implicated in discordance between AI-predicted risk scores and laboratory calculated risk scores. The additional training from the select sample provides a correction model ensuring the AI-predicted disease risk is highly accurate and precise. AI systems of the invention and classify breast cancer risk from WSIs of stained breast tissue with sensitivity and specificity rivaling prior microarray assays.

### TECHNICAL FIELD

The invention relates to digital pathology systems that use artificial intelligence or machine learning to evaluate disease risk.

### BACKGROUND

Breast cancer is the second most common cancer among women in the United States. Despite advances in screening and treatment, breast cancer remains the second leading cause of cancer death among women. Further, recent studies have shown that there are racial/ethnic variations in breast cancer tumor characteristics, subtypes, relative treatment success rates, and recurrence rates. Moreover, the efficacy of various treatments diverges amongst breast cancer subtypes at various stages of progression. This creates a complex picture for pathologists and oncologists in diagnosing, treating, and predicting recurrence in breast cancer patients. Thus, while the aggregate impact of breast cancer is clear, accurate, patient-specific diagnosis, prognosis, and treatment remains relatively obscured.

Although breast cancers come in various forms and presentations, they are generally classified based upon histological appearance, i.e., using histopathological practices. This generally means a biopsy followed by a microscopic image analysis. In doing so, a pathologist will often examine whole-slide images (WSI) of tissue obtained from the biopsied tissue to type and stage the malignancy. This is a time consuming and difficult process.

Not only are these histopathology assays difficult, but they are often inconsistent due to the “human factor”. Although systematic training and guideline harmonization have been implemented, histopathology relies on the subjective analysis, visual perception, and judgment of individual pathologists.

### SUMMARY

The invention provides methods and artificial-intelligence (AI) (including Machine Learning systems) that predict disease risk from samples. In particular, AI systems predict diseases risks from digital samples, such as scanned whole slide images (WSIs) or partial scans of cellular material. When those AI-predicted risks do not match risk scores calculated in laboratory assays, methods of the invention selected relevant additional samples for training and/or give the AI system additional training on (e.g., the) select samples. In particular, the samples for additional training are selected or created for their representation of biological processes implicated in discordance between AI-predicted risk scores and laboratory calculated risk scores.

After an AI model is initially trained, concordance is measured between the risks calculated from e.g., the laboratory assay (e.g., microarray) and risks predicted by the trained AI model. Preferably, concordance is evaluated on a per-patient basis. For each patient, a determination may be made whether the results given by conventional lab assay are concordant with the result given back by AI-based predications. When investigating the biology (biological processes), it has been observed that lab assays (such as microarrays) perform differently in some biological processes from AI-based detections. Those biological processes for which both methods perform differently are identified. Additional samples that exhibit those biological processes are obtained. For example, specific biological phenomenon may subject to IHC staining. And those additional samples are then used to improve the AI training. Improving the AI training may include either or both (i) providing the additional samples as training inputs to the trained AI model or (ii) by using those additional samples for training another, additional AI model (of a similar or different type). Whether using either or both approaches to improving the AI training, the resultant trained model(s) may be referred to as a correction model.

The additional training from the select samples provides a correction model ensuring that predicted risks by the AI system are highly accurate and precise. AI systems of the invention thus classify breast cancer risk from images of stained breast tissue with sensitivity and specificity rivaling or surpassing prior laboratory gene-expression assays.

AI systems of the invention may include any suitable machine learning (ML) algorithm. For example, a system may use an ML algorithm such as neural network, a random forest, an adaptive boosting algorithm, a support vector machine, others, or combinations thereof. Training a ML learning algorithm of the invention may be supervised, unsupervised, or weakly supervised. In preferred embodiments, the AL system uses a convolutional neural network (CNN) as a classifier that is trained in a weakly-supervised method known as multiple instance learning (MIL). For MIL, the CNN is trained on whole slide images (WSIs) that are presented as bags, where each bag comprises a plurality of tiles from one slide and a diagnostic label from that slide. The CNN evaluates each tile to identify those tiles most suspicious of being informative of a positive disease diagnostic. In some embodiments, the CNN passes a number of most suspicious tiles from each slide to a recurrent neural network (RNN) aggregator that aggregates the predicted risks for each tile and outputs a predicted disease risk score for the whole slide. Those predicted risk scores are generated for an arbitrarily large training data set (e.g., thousands of slides) and compared to known risk scores calculated by an assay such as MammaPrint (MP). Where the predicted risk scores correlate weakly with the calculated risk scores, the correction model described herein is applied to subject the AI system to additional training to improve its predictions.

The provided “correction model” addresses biological processes that are valuated quantitatively differently between the AI system and a laboratory test. Those biological processes may be identified by tests such as differential gene expression analysis on groups of samples associated with lack of congruence between digital predictions and lab-calculated risk scores. Groups of samples exhibiting those biological processes are selected and used in additional training of the AI system, thereby improving the predictions made by the AI system.

Thus, methods and systems of the invention benefit from a correction model that identifies biological processes implicated in discordance between predicted- and calculated risk scores. The correction model involves providing additional training to the AI system, by giving new training input to the AI model or by training a new, additional AI model, using samples that exhibit those identified biological processes or samples in which those biological processes are emphasized (e.g. IHC staining). For example, the samples may be digital images of histopathology slides, and an AI system may use deep machine learning to make risk predictions from those slides. In parallel, laboratory results, such as the MammaPrint 70-gene signature panel, may be used to calculate breast cancer risk. The AI system may predict a disease risk for breast cancer based on whole-slide images (WSI) of breast tissue. For samples for which the predictions are discordant with the MammaPrint results, at least one differentially active biological process may be identified. For example, in numerous of such cases, a process such as mitosis or extracellular matrix remodeling may be highly active. Additional WSIs that represent the (IHC stained) mitosis, or the extracellular matrix remodeling, or other such process are presented back to the AI system for additional training. With implementation of the described correction model, the AI system will give disease risk predictions that are highly sensitive, specific, and concordant with calculated risk scores.

In certain aspect, the invention provides pathology diagnostic methods. Preferred methods include providing samples from a plurality of subjects as inputs to an AI system; operating the AI system to output predicted disease risks for the subjects; and correlating the predicted disease risks to calculated risk scores from biomarker data from the subjects. A subset of the samples having weak correlation between the predicted disease risks and the calculated risk scores is selected and a correction model is implemented that further involves identifying a biological process that is differentially active in the subset compared to at least a second subset of the samples and training the AI system (e.g., the same AI/ML model with further training or an additional AI/ML model of similar or different type to be used within the AI system) an additional samples known to represent the biological process. The method may include creating those additional samples, e.g., by staining slides for the biological process.

The samples may be images of stained tissue slices, such whole slide images of stained tissue sections. In some embodiments, the AI system comprises a deep neural network or convolutional neural network. Prior to the operating step, the neural network may have been trained using multiple instance learning from training data comprising (i) whole slide images (WSIs) of slides, where each slide is presented as a bag comprising multiple tiles of pixels from that slide, and (ii) a diagnostic outcome for each slide. In certain embodiments, the deep neural network selects informative tiles from each slide (e.g., tiles most suspicious of being informative of a diagnosis of disease) and passes features from the informative tiles for each slide to a neural network that aggregates the informative tiles and predicts a final slide-level classification for that slide.

As mentioned, methods of the invention include correlating predicted disease risks from the AI system to calculated risk scores from biomarker data from the subjects. The biomarker data may be gene expression data obtained by, e.g., a microarray or RNA sequencing. The selected subset may be those samples sharing a defined range of the predicted disease risks or the calculated risk scores. The selecting step may further involve performing differential expression analysis for samples of the subset to identify genes differentially expressed implicated in the weak correlation of the subset. The method may include identifying the biological process based on the identified genes. For example, the identified gene and the biological process may be a pair such as FLT1 and proliferation, or COL4A2 and tumor invasion, STAT1 and immune response, FGF18 and angiogenesis, or BBC3 and dysregulated apoptosis.

The samples may be whole slide images (WSIs) and the AI system may divide each WSI into tiles, score the tiles, identify one of the highest- and lowest-scoring tiles for each slide. The AI system may further calibrate scores for the remaining tiles based on the identified one of the highest- and lowest-scoring tiles for that slide. In some embodiments, the method includes after the training step, using the AI system to identify a risk of cancer (e.g., breast cancer) for a patient from at least one test image of tissue from the patient.

In related aspects, the invention provides a digital pathology system that includes a computer system comprising at least one processer coupled to memory having an AI system resident therein wherein the system is operable to perform the described methods.

### BRIEF DESCRIPTION OF THE DRAWINGS

FIG. 1 is a plot of predicted risk index versus calculated risk index.

FIG. 2 shows organization of an AI system according to certain embodiments.

FIG. 3 illustrates vertical comparisons to identify groups.

FIG. 4 illustrates identifying a group by a horizontal comparisons.

FIG. 5 shows samples that represent a biological process used in additional training.

### DETAILED DESCRIPTION

The disclosure provides systems and methods useful in pathology for determining risk of a disease or condition. In particular, systems and methods of the disclosure use an artificial intelligence or machine learning system (AI system), that has been trained on digital samples such as scanned, digital images of histopathology slides. The AI system predicts a risk of disease from those digital inputs. The AI system may also be given inputs in the form of diagnostic or disease risk calculations from biological data such as biomarker data. For example, the AI system may be given a digital input (e.g., a scanned slide) and a laboratory diagnostic result (e.g., a disease-risk score from gene expression data) for the subject. The AI system correlates the digitally-predicted disease risk to the disease risk calculated from the laboratory data.

In exemplary embodiments for the detection of breast cancer, an AI system of the disclosure predicted a breast cancer risk index from digitized whole slide images (WSIs) of a set of 4191 samples. For those samples, the system also had a disease risk index calculated from microarray data as well as full-genome microarray data.

Table 1 shows the number of samples that were initially correctly/incorrectly classified by a system according to certain embodiments before a retraining operation of the disclosure.

TABLE 1
Predicted and calculated risk classification.

| | High-Risk predicted | Low-Risk predicted |
|---|---|---|
| | by AI system and WSI | by AI system and WSI |
| High-Risk calculated | 1431 | 626 |
| from expression data | | |
| Low-Risk calculated | 300 | 1834 |
| from expression | | |

The disclosure provides methods and system to decrease the number of those 926 weakly-corrected samples, i.e., to improve correlation for the 626 entries associated with High-Risk calculated from expression data and Low-Risk predicted by AI system and WSI, and the 300 entries that were associated with Low-Risk calculated from expression data and High-Risk predicted by AI system and WSI. Specifically, methods and system of the disclosure use methods of training, and re-training (or continuing to train) an AI system to minimize weakly correlated samples, i.e., to improve concordance between AI predictions and lab-test calculated risk scores.

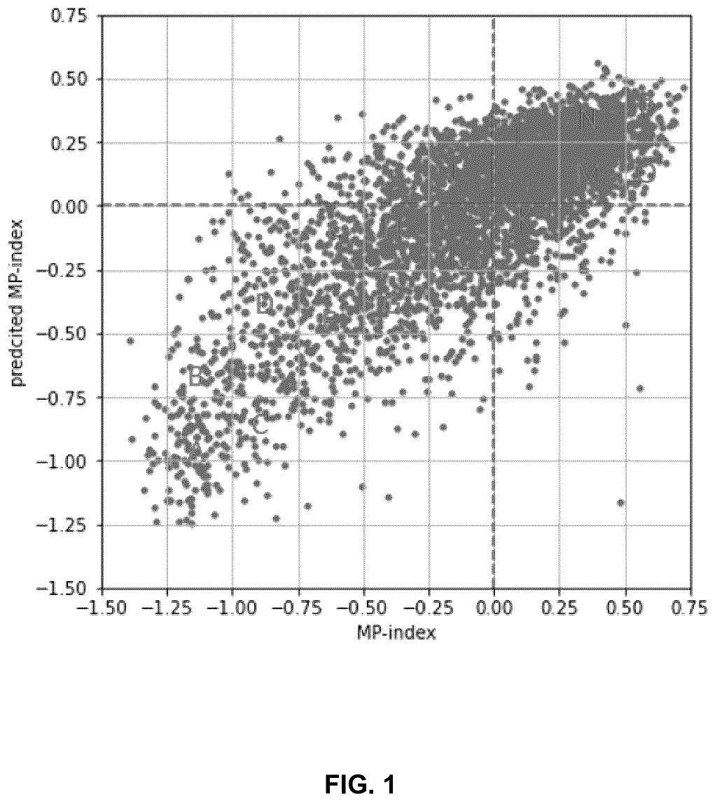

Table 1 depicts an exemplary binary classification setup in which risk is calculated or predicted to be high or low. Systems and methods of the invention may categorize risk within a plurality of buckets, or may preferably even directly predict risk index scores (e.g., along a numerical range). For example, as shown in FIG. 1, “MP-index” refers to a numerical index value calculated from a laboratory assay (such as the MammaPrint microarray) and “predicted MP-index” refers to a specific index value given to a patient by an AI model.

FIG. 1 is a scatter plot showing the AI-system predicted risk index versus calculated risk index. The plotted data include indices (on the x-axis) calculated by microarray analyses of expression of 70 genes in a test sold under the trademark MAMMAPRINT (MP) by Agendia NV (Amsterdam, The Netherlands). The MP test is described in Cardoso, 2016, 70-gene signature as an aid to treatment decisions in early-stage breast cancer, NEJM 375(8):717-729, incorporated by reference. Calculated risk scores from the 70 gene microarray are presented along the x-axis, labeled MP-index. The y-axis, labeled “predicted MP-index” gives risk scores predicted by an AI system of the disclosure.

The AI-system may include any suitable one or combination of an artificial intelligence (AI) or machine learning (ML) model. One example is illustrated here, but any suitable model may be included. Suitable AI/ML models for use in an AI system of the invention may include, for example, random forests, a support vector machine (SVM), boosting algorithms (e.g., adaptive boosting (AdaBoost), gradient boost method (GSM), extreme gradient boost methods (XGBoost)), neural networks such as H2O, deep-learning networks, and/or specific implementations thereof including deep neural networks, convolutional neural networks, recurrent neural networks, and combinations thereof. Those AI/ML models may each be used alone or combined in any suitable combination. What is important is that an AI system predict risks that are correlated to assay-predicted risks. For samples with low concordance between the assay and the AI-prediction, a differentially active biological process is identified, and additional samples representing that biological process are used for further training, e.g., on another AI/ML model or for additional training on the AI/ML model.

In an exemplary embodiment presented here, the AI system uses multiple instance learning with convolutional neural network as a classifier and a recurrent neural network as an aggregator. Any other suitable setup, machine learning model, combination of models, training process, or AI architecture may be used. The given examples illustrate the principle.

The AI system includes a multiple instance learning network that operates on whole slide images (WSI) presented as bags of sections of the WSI with a positive/negative diagnosis from the slide given for each bag. The AI system includes a recurrent neural network (RNN) that aggregates the “most suspicious” sections (i.e., for each slide, the sections with the highest risk scores are passed to the RNN) and makes a final slide level classification, or “predicted MP-index” for each slice.

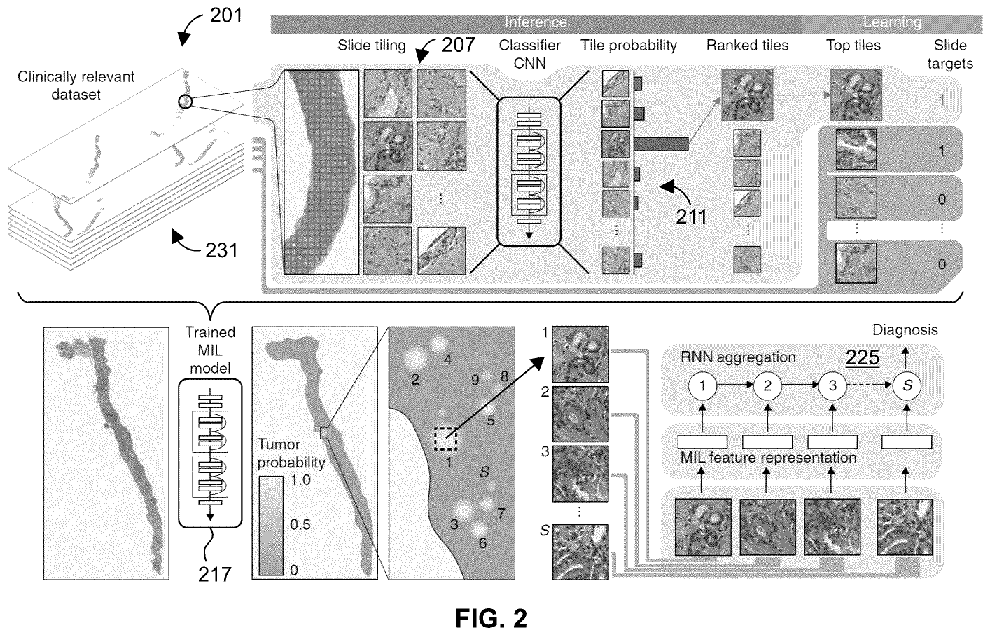

FIG. 2 shows organization of an AI system according to certain exemplary embodiments. The top of the figures shows a series of steps involved in training a classier 217. The bottom of the figures illustrates how test samples are presented to the classifier 217 and aggregator 225.

For training, and again later in-use, samples 201 are presented to a classifier 217. In the described embodiment, the classifier 217 is a convolutional neural network (CNN) that operates by a weakly-supervised learning process and is trained by multiple instance learning (MIL). In MIL, each training data slide is presented as a bag 207 of tiles 211 (e.g., of 224 pixels by 224 pixels) and each bag 207 gets the diagnostic label from that slide.

During training (top of figure), the classifier makes a full pass through the training data set and ranks the tiles according to their probability of being positive. The CNN learns on the top-ranking tiles per bag 207 to yield the trained MIL model as the classifier 217. For diagnosis, i.e., to output the predicted MP-index, aggregation will be performed at the slide level with a recurrent neural network (RNN) as an aggregator 225. The output of the classifier 217 for some number, s, of most suspicious tiles from each slide, is passed to the aggregator 225, which outputs a predicted risk score. Certain embodiments include a training strategy to give optimal results in which the classifier identifies a high- and low-scoring tile for a slide, and calibrates the scoring of the other tiles of that slide based on the high- and low-scoring tile. For example, where the samples comprise whole slide images (WSIs), and the AI system divides each WSI into tiles, scores the tiles, identifies one of the highest- and lowest-scoring tiles for each slide, and calibrates scores for the remaining tiles based on the identified one of the highest- and lowest-scoring tiles for that slide. This embodiment, which may be dubbed “high-, low-calibration”, may be found to give best results in the disclosed model of multiple instance learning, in which each slide is presented to the classifier as a bag of tiles.

Once the classifier 217 is trained, it may be operated on samples 201 to output the predicted risk scores that are correlated to the calculated risk scores (see FIG. 1). In one features of the invention, additional samples 231 are then used to further train, or re-train, the classifier 217, after the predicted risk scores are correlated to calculated scores from laboratory analysis of biomarkers.

Most preferably, the additional samples are based on correlating predicted risk scores to calculated risk scores, identifying samples with weak correlation, identifying (e.g., by differential expression analysis) a biological process implicated in the weak correlation, and further training the classier 217 on additional samples 231 that exhibit the biological process.

In FIG. 1, each dot in the scatter plot shows the predicted MP-index and the MP-index for one sample. If the AI system were perfectly concordant with the results from the biomarkers (gene expression by microarray), all of the dots would plot along the diagonal. The fact that the plot includes some samples that plot off of the diagonal may be interpreted to mean that there is good concordance between the AI system and the biomarkers but that there is an opportunity for improvement.

The disclosure provides methods for improving the training of the AI system. A subset of the plotted samples is addressed for further analysis. Specifically, in some embodiments, the scatter plot is used to select different groups of samples which are then subject to differential expression analysis (limma package in R-software may be used to perform the analysis).

Different strategies may be used to identify subsets, which represent groups of samples that include at least some for which MP index and predicted MP index are weakly correlated. Those groups may be identified or selected by taking a set of samples that lie within a narrow range of MP index in the plot (“vertical” groups) or a set of sample that lie within a narrow or defined range of predicted MP index (“horizontal” groups).

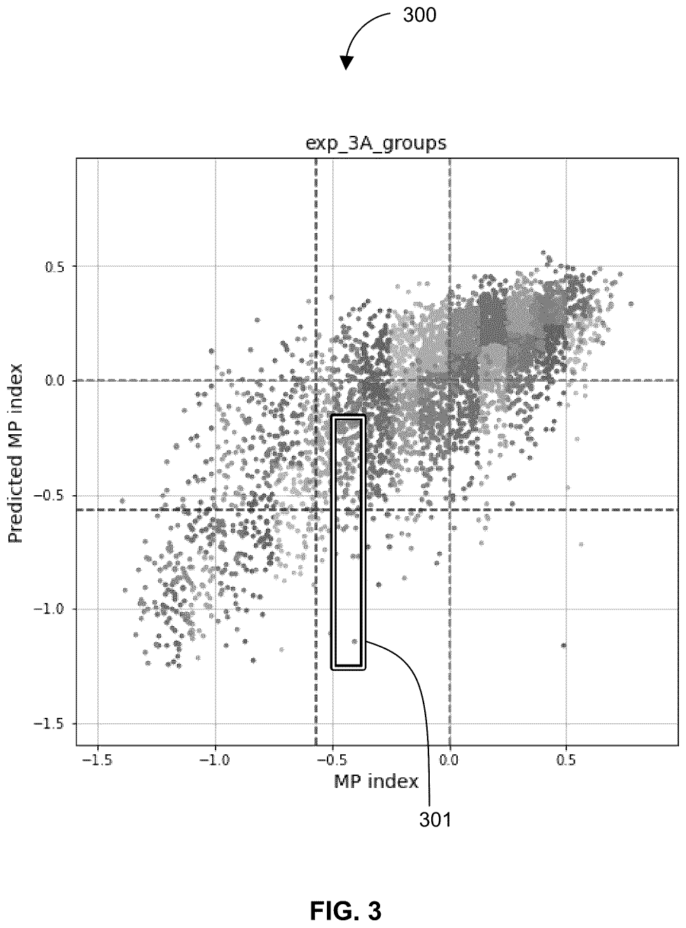

FIG. 3 illustrates vertical comparisons. As shown in the vertical comparison scatter plot 300, the groups are made by moving along the MammaPrint (MP) index axis so that the MammaPrint indices are in the same range for the samples in vertical groups. In the plot, one exemplary subset 301 is called out to illustrate. The subset 301 is defined as those samples for which the MP index lies between some values such as −0.5 and −0.375. In this example, the upper boundary of predicted MP index for the subset 301 is the center of the MP index range, −0.4375. The lower boundary of predicted MP index is arbitrary (e.g., −1.25 or some value that simply includes all samples below the diagonal within the MP index range (−0.5 through −0.375). Any suitable variation of these values may be used. An important feature is that the subset includes samples that are off-axis, which are samples for which predicted MP index and MP index are poorly concordant. This group may be used for “vertical comparisons', which refers to how the subset 301 appears when a bounding box is drawn on the plot. The invention may also use horizontal comparison.

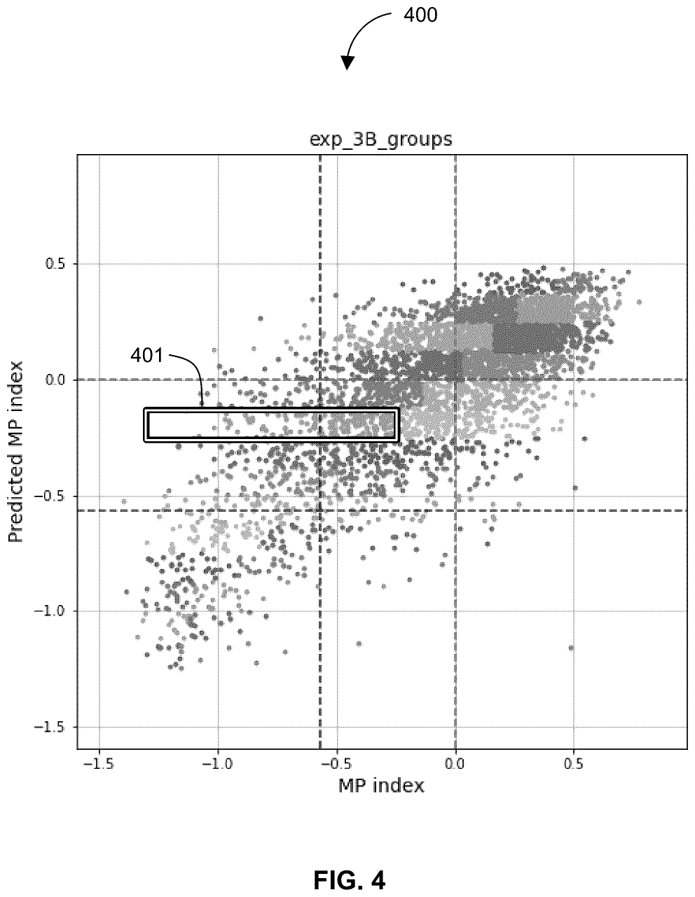

FIG. 4 illustrates selecting a subset 401 in embodiments that may be referred to as “horizontal comparisons”. As shown in the horizontal comparison scatter plot 400, the groups are made by moving along the predicted MammaPrint-index axis. A box is drawn to illustrate selection of one potential subset 401, which includes samples for which the predicted MP index lies between some certain values such as −0.125 and 0.25. The right boundary of the box may be the MP index value for the center of that range, i.e., MP index=0.1875. The left boundary may be arbitrary (e.g., MP index=−1.25) so that all samples to the left of the right boundary are included. But, by selecting the subset 401 in this manner, all samples in the subset have predicted MP index values within a defined, limited range.

Methods of the invention may include analyzing samples in one or more of the subsets 301, 401 to identify a biological process at play in those samples that are “off-axis”, i.e., those samples for which predicted risk scores and calculated risk scores are weakly correlated.

Any suitable method may be used to identify biological processes exhibited in those samples. For example, whole slide images from those samples may be presented to histopathologists for review and labeling. In another example, microarray results from a MammaPrint assay may be manually reviewed and annotated. In certain embodiments, those samples are subject to differential gene expression analysis. Any suitable method may be used for differential gene expression analysis. For example, a sample may be subject to gene expression analysis by a next-generation sequencing (NGS) technology including, for example, by single-cell RNA sequencing. See Wang, 2019, Comparative analysis of different gene expression analysis tools for single-cell RNA sequencing data, BMC Bioinformatics 20: article 40, incorporated by reference. In some embodiments, a microarray assay is analyzed for differential expression. For example, a human transcriptome array (e.g., using a Affymetrix GeneChip) may be used to analyze mRNA expression in a tissue sample, and an RVM t-test may be applied to filter differentially expressed mRNAs, to identify mRNAs that are significantly up- or down-regulated (fold change>1.2, p<0.05) in the tissue compared with the adjacent noncancerous tissue. See Pan, 2017, Analysis of differential gene expression profile identifies novel biomarkers for breast cancer, Oncotarget 8(70):114613-114625, incorporated by reference.

Differential expression analysis may be performed on two subsets of samples to identify genes differentially expressed that are implicated in the weak correlation of samples of the subsets. When a gene (or biological group) is found differentially expressed between two horizontal groups (subsets), that may be referred to as a horizontal differential. Here, identifying a biological process includes performing differential expression analysis to identify a process that is differentially active in one horizontal subset compared to a second horizontal subset. Those two horizontal subsets may be taken from within one bin of AI-predicted disease risks. Similarly, vertical groups may be used, where first and second subsets are taken from within a “bin”, or determined range, of calculated disease risks. Whether using horizontal or vertical groups, the differential expression analysis reveals differentially expressed genes that are implicated in lower concordance.

Those identified, differentially expressed genes may be used to identify a biological process. Specific biological processes at play may include an acquired resistance to apoptosis, disrupted antigrowth signaling, altered expression of growth factors, proliferation and oncogenic transformation, uncontrolled cells cycle, altered extracellular matrix adhesion and remodeling, gain of motility or actin filament re-organization, altered metabolism, or altered expression of angiogenesis effectors, for example. In fact, genes that are found by differential expression analysis to be differentially expressed in those subsets that include samples that are weakly correlated (i.e., discordant, or off-axis in the plots) may be used to look up a biological process in a reference source or database. Biological process of certain genes that may be differentially expressed are described in sources such as Tian, 2010, Biological functions of the genes in the Mammaprint breast cancer profile reflect the hallmarks of cancer, Biomarker Insights 5:129-138, incorporated by reference.

Using such methods on the samples in the figures, 25 genes were found that appear in both of the horizontal and vertical differential expression gene analysis. Out of these 25 genes, there are 24 genes that are involved in cell cycle, mitosis, and cell-division processes (DAVID functional annotation clustering is used). From the horizontal analysis, we found 28 genes that are differentially expressed (adj. p-value<0.05) and all belong to either Extracellular matrix or Glycoprotein clusters.

An insight of the invention is that a specific biological process may be implicated in discordance between AI predictions and calculated test scores, that some subset of the samples with weak correlation may be analyzed to identify genes or molecular pathways implicated in the week correlation, and a biological process associated with the implicated genes or molecular pathways may be identified and used for advanced training of the AI system. Specifically, the AI system may be trained on additional samples known to represent the biological process.

Additionally, systems and methods of the invention are useful specifically to improve the concordance along one axis such as, e.g., the “MP-index” axis in FIG. 1. This means that concordance may specifically be improved for a specific group (such as for a specific risk-group such as ultra-low, or low, or high . . . etc.) by specifically identifying the biological processes at play at the relevant range of index scores for the specific group. That is moving along the calculated index axis, systems and methods of the invention are useful to improve concordance for a certain group, or category of risk scores, from e.g., the calculated risk scores. This holds true for any clinically relevant subset. For example, where calculated risk scores (“MP-index”) fall into an ultra-low risk category, systems and methods of the invention are useful to improve concordance within that category, or range of MP-index scores.

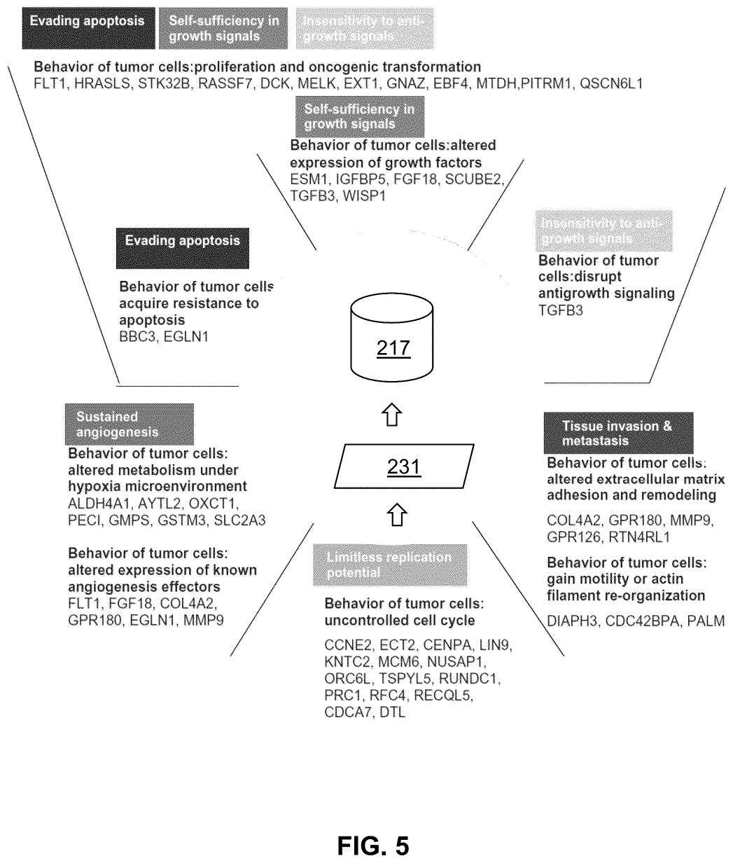

FIG. 5 is a cartoon to show how a biological process may be identified from differentially expressed genes and providing additional samples 231 that represent the biological process to the classifier 217 for additional training of the classifier 217 in the AI system. Any suitable analytical system or software package may be used. For example, when performing differential expression analysis, a common denominator of the differentially expressed genes may be identified after comparing to (horizontal or vertical) gene groups. Some embodiments may use the gsEasy software package, which includes a function “gset” for calculating p-values of enrichment sets of genes and a list of gene ontology (GO) annotations. The gsEasy package implements methods described in Subramanian, 2005, Gene set enrichment analysis: a knowledge-based approach for interpreting genome-wide expression profiles, PNAS 102(43):15545-1550, incorporated by reference. As shown by the cartoon in FIG. 5, differential expression analysis is performed on samples of two of subset 301 or 401 and one or more differentially-expressed genes such as CCNE2, ECT2, CENPA, LIN9, KNTC2, MCM6, NUSAP1, ORC6L, TSPYL5, RUNDC1, PRC1, RFC4, RECQL5, CDCA7, or DTL may be identified.

Because those genes are understood to participate in an uncontrolled cell cycle, additional samples 231 that exhibit mitosis are selected for additional training of the AI system.

The additional samples 231 may be selected by any suitable process. For example, in some embodiments, samples are whole slide images that have been curated and labeled by expert pathologists. Those samples may make up an archive (e.g., measuring in terrabytes to petabytes) of digital files, each of which may be a whole slide image. Each file may already be labeled (for the whole slide or at tiles or regions within) by the expert pathologists for features such as mitotic spindle/mitosis, apoptotic bodies/apoptosis, stained angiogenic factors/angiogenesis, actin content/metastasis, or others. From the differentially-expressed genes identified in the subset(s) 301, 401, the system can query labels for the samples (e.g., electronically query a database such as the archive) to identify the additional samples 231 that have been labelled as representing the biological process. Those additional samples 231 are then presented to the classifier 217 of the AI system for additional training of the classifier 217.

Because the classifier 217 is given additional training on samples that exhibit the biological processes that were detected to be active in results associated with discordant results between the AI system predictions and the scores calculated from the biomarker assay (e.g., the 70-gene digital signature obtained by the microarray-based Mammaprint test), the classifier 217 will exhibit improved concordance after the additional training.

Any suitable system may be used in the AI system. For example, in the examples given above, the classifier 217 uses a convolutional neural network (CNN) and the aggregator 225 uses a recurrent neural network. A neural network (NN) is a specific example of a machine learning (ML) or artificial intelligence (AI) tool. A NN is based on a collection of connected units or nodes called artificial neurons, which loosely model the neurons in a biological brain. Each connection, like the synapses in a biological brain, can transmit a signal to other neurons. An artificial neuron receives a signal then processes it and can signal neurons connected to it. The “signal” at a connection is a real number, and the output of each neuron is computed by some non-linear function of the sum of its inputs. The connections are called edges. Neurons and edges typically have a weight that adjusts as learning proceeds. The weight increases or decreases the strength of the signal at a connection. Neurons may have a threshold such that a signal is sent only if the aggregate signal crosses that threshold. Typically, neurons are aggregated into layers. Different layers may perform different transformations on their inputs. Signals travel from the first layer (the input layer) to the last layer (the output layer), possibly after traversing the layers multiple times. A NN is instantiated in computer hardware and takes digital information as inputs and gives a digital output. Neural networks may be trained in a supervised, unsupervised, or semi-supervised fashion.

Neural networks, modeled on the human brain, allow for processing of information and machine learning. Systems and methods of the invention may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 80 Million Gallery, 2015), each incorporated by reference.

Deep learning neural networks (also known as deep structured learning, hierarchical learning or deep machine learning) include a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower level features to form a hierarchical representation. Those features are preferably represented within nodes as feature vectors. Deep learning by the neural network includes learning multiple levels of representations that correspond to different levels of abstraction; the levels form a hierarchy of concepts. In some embodiments, the neural network includes at least 5 and preferably more than ten hidden layers. The many layers between the input and the output allow the system to operate via multiple processing layers. Neural networks that include a convolutional layer are included in embodiments of the disclosure.

The CNN classifier 217 described above is trained by multi-instance learning, which is a type of semi-supervised learning. In multi-instance learning, input is presented in bags with a label for each bag. In these cases, each bag includes tiles taken from one digitized histopathology slide, and the label is a diagnosis for that slide.

A convolutional neural network (CNN) is a neural network that employs a mathematical operation called convolution. Convolutional networks are a specialized type of neural networks that use convolution in place of general matrix multiplication in at least one of their layers. In a CNN, the input is a tensor with a shape: (number of inputs)×(input height)×(input width)×(input channels). After passing through a convolutional layer, the image becomes abstracted to a feature map, also called an activation map, with shape: (number of inputs)×(feature map height)×(feature map width)×(feature map channels). Convolutional layers convolve the input and pass its result to the next layer. This is similar to the response of a neuron in the visual cortex to a specific stimulus. Each convolutional neuron processes data only for its receptive field. Convolutional networks may include local and/or global pooling layers along with traditional convolutional layers. Pooling layers reduce the dimensions of data by combining the outputs of neuron clusters at one layer into a single neuron in the next layer. Local pooling combines small clusters, tiling sizes such as 2×2 are commonly used. Global pooling acts on all the neurons of the feature map. There are two common types of pooling in popular use: max and average. Max pooling uses the maximum value of each local cluster of neurons in the feature map, while average pooling takes the average value.

Recurrent neural networks (RNN) are a class of artificial neural networks where connections between nodes form a directed or undirected graph along a temporal sequence. This allows it to exhibit temporal dynamic behavior. Derived from feedforward neural networks, RNNs can use their internal state (memory) to process variable length sequences of inputs. The term “recurrent neural network” is used to refer to the class of networks with an infinite impulse response, whereas “convolutional neural network” refers to the class of finite impulse response.

Here, having separate aggregator 225 after classifier 217 mitigates issues with max pooling in the CNN associated with false positive in the whole slide classification. The aggregator may also suitably be a random forest, trained on manually engineered features extracted from a heat map generated by the MIL-based tile classifier 217. Given a vector representation of tiles within the MIL classifier (even if singularly they were not classified as positive by the tile classifier), taken together the tiles could be suspicious enough to trigger a positive response by a representation-based slide-level classifier. Based on those ideas, an RNN-was selected as aggregator 225 to integrate information and provide the final slide classification.

Convolutional neural networks (CNNs) and recurrent neural networks (RNNs) both implement examples of machine learning generally understood to include deep learning.

Deep learning is part of a broader family of machine learning methods based on learning representations of data. An observation can be represented in many ways such as a vector of intensity values per pixel, or in a more abstract way as a set of edges, regions of particular shape, etc. Those features are represented at nodes in the network. Preferably, each feature is structured as a feature vector, a multi-dimensional vector of numerical values.

Any machine learning algorithm may be used in an AI system of the invention include, for example, a random forest, a support vector machine (SVM), or a boosting algorithm (e.g., adaptive boosting (AdaBoost), gradient boost method (GSM), or extreme gradient boost methods (XGBoost)), or neural networks such as H2O.

Machine learning algorithms generally are of one of the following types: (1) bagging (decrease variance), (2) boosting (decrease bias), or (3) stacking (improving predictive force). In bagging, multiple prediction models (generally of the same type) are constructed from subsets of classification data (classes and features) and then combined into a single classifier. Random Forest classifiers are of this type. In boosting, an initial prediction model is iteratively improved by examining prediction errors. AdaBoost and extreme Gradient Boosting are of this type. In stacking models, multiple prediction models (generally of different types) are combined to form the final classifier. These methods are called ensemble methods. The fundamental or starting methods in the ensemble methods are often decision trees. Decision trees are non-parametric supervised learning methods that use simple decision rules to infer the classification from the features in the data. They have some advantages in that they are simple to understand and can be visualized as a tree starting at the root (usually a single node) and repeatedly branch to the leaves (multiple nodes) that are associated with the classification.

In some embodiments, an AI system of the invention uses a random forest. The random forest uses decision tree learning, where a model is built that predicts the value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable.

SVMs can be used for classification and regression. When used for classification of new data into one of two categories, such as having a disease or not having a disease, a SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. SVMs can also be used in support vector clustering. See Ben-Hur, 2001, Support Vector Clustering, J Mach Learning Res 2:125-137, incorporated by reference.

Boosting algorithms are machine learning ensemble meta-algorithms for reducing bias and variance. Boosting is focused on turning weak learners into strong learners where a weak learner is defined to be a classifier which is only slightly correlated with the true classification while a strong learner is a classifier that is well-correlated with the true classification. Boosting algorithms consist of iteratively learning weak classifiers with respect to a distribution and adding them to a final strong classifier. The added classifiers are typically weighted in based on their accuracy. Boosting algorithms include AdaBoost, gradient boosting, and XGBoost. See Freund, 1997, A decision-theoretic generalization of on-line learning and an application to boosting, J Comp Sys Sci 55:119; and Chen, 2016, XGBoost: A Scalable Tree Boosting System, arXiv: 1603.02754, both incorporated by reference.

In most preferred embodiments, whatever machine learning algorithm is used (e.g., neural network, random forest, SVM, etc.), the AI system uses the machine learning algorithm to predict disease risk scores from the sample. The AI system correlates those predicted risk scores with scores calculated from a laboratory assay of biomarkers. For example, the biomarker data may include gene expression data. Gene expression data may be obtained by microarray (e.g., MammaPrint) or RNA sequencing. Methods involve selecting a subset of samples that include some samples with weak correlation (poor concordance) between predicted and calculated risk scores. That subset is analyzed to identify genes and a biological process differentially active in the samples with weak correlation (i.e., what biological process and genes appear to underly the weaker correlations). For example (making reference to FIG. 5), the identified biological process and identified genes include a pair such as: proliferation and FLT1; tumor invasion and COL4A2; immune response and STAT1; angiogenesis and FGF18; or apoptosis and BBC3.

From samples such as those samples used in training the AI system, additional samples are selected that exhibit evidence of the selected biological process. For example, where the samples comprise whole slide images (WSIs), those WSIs may be stored in a database or archive a slide-level or tile-level annotations for the visual hallmarks of, e.g., proliferation, tumor invention, deviant immune response, angiogenesis, or deviant apoptosis. Some of those samples, a subset, maybe presented back the AI system for additional training. By further training the AI system with sample selected for exhibiting those processes implicated in discordant predications, the invention provides AI systems with high sensitivity and specificity and predicting disk risk, and which AI systems make disease risk predictions that are highly concordant with disease risk calculated from laboratory assays of biomarkers.

Methods of the disclosure may be performed using a system of the disclosure. Some embodiments provide a hardware computer system with at least on processor coupled to memory having an AI system resident therein. The system has instructions stored therein executable by the processor to cause the system to perform methods of the disclosure.

Preferably the system is operable to use the AI system to output predicted disease risks for subjects from samples provided as inputs to the system. They system may further be operable to correlate the predicted disease risks to calculated risk scores from biomarker data from the subjects (e.g., as shown in FIG. 1). The system may be used to select a subset 301, 401 of the samples having weak correlation between the predicted disease risks and the calculated risk scores. The system may be used to identify a biological process that is differentially active in the subset compared to all of the samples (e.g., via a lookup in a database of such relationships as those shown in FIG. 5) and, as shown in FIGS. 5 and 2, additional samples 231 known to represent the biological process are presented to the AI system to further train a classifier such as the classifier 217.

The invention provides methods and artificial-intelligence (AI) systems that predict disease risk from samples. In particular, AI systems predict diseases risk from samples such as whole slide images (WSIs). When those AI-predicted risks do not match calculated risk scores from laboratory assays, methods of the invention give the AI system a pre-selection algorithm for selecting additional samples for further, additional training and/or further, additional training on select samples. In particular, the samples for additional training are selected for biological characteristics found in WSIs such as their representation of biological processes implicated in discordance between AI-predicted risk scores and laboratory calculated risk scores. When there is discordance between AI-predicted risk scores and laboratory calculated risk scores, methods and systems may select groups of samples within which differentially active biological processes are implicated in the discordance. Using methods of the invention, AI systems are trained to predict disease risk, and undergo additional, further training on select samples so that predictions are accurate and precise. In particular, methods are given for addressing a lack of concordance between digital, or AI system, risk predictions and risk predictions calculated from laboratory assays such as gene expression analyses. Methods of the disclosure provide a “correction model”, a methodological process for addressing lack of congruence between digital predictions and lab-calculated risk scores. Methods involve identifying biological processes that are valuated quantitatively differently between an AI system and results from a laboratory tests. Those biological processes may be identified by tests such as differential gene expression analysis on group of samples associated with lack of congruence between digital predictions and lab-calculated risk scores. Groups of samples exhibiting those biological processes are selected and used to further train, or re-train, an AI system, thereby improving the predictions made by the AI system.

1. A pathology method comprising: providing samples from a plurality of subjects as inputs to an AI system; operating the AI system to output predicted disease risks for the subjects; correlating the predicted disease risks to calculated risk scores from biomarker data from the subjects; selecting a subset of the samples having a correlation between the predicted disease risks and the calculated risk scores; identifying a biological process that is differentially active in the subset compared to at least one other subset; and training the AI system on additional samples known to represent the biological process.

2. The method of claim 1, wherein the samples comprises images of stained tissue slices.

3. The method of claim 1, wherein the samples comprise whole slide images of stained tissue sections.

4. The method of claim 1, further comprising obtaining the additional samples by staining additional slides for the biological process.

5. The method of claim 1, wherein the AI system comprises a deep neural network.

6. The method of claim 5, wherein the training step comprises: providing further training input into the deep neural network; or training a second machine learning model using the additional samples.

7. The method of claim 5, wherein prior to the operating step the deep neural network is trained by multiple instance learning from training data comprising (i) whole slide images (WSIs) of slides, where each slide is presented as a bag comprising multiple tiles of pixels from that slide, and wherein each slide; and (ii) a diagnostic outcome for each slide.

8. The method of claim 6, wherein the deep neural network selects informative tiles from each slide and passes features from the informative tiles for each slide to a neural network that aggregates the informative tiles and predicts a final slide-level classification for that slide.

9. The method of claim 1, wherein the biomarker data comprises gene expression data.

10. The method of claim 9, wherein the gene expression data is obtained by microarray or RNA sequencing.

11. The method of claim 1, wherein the selecting step comprises selecting the subset as being those samples sharing a defined range of the predicted disease risks or the calculated risk scores.

12. The method of claim 1, wherein the selecting step further comprises performing differential expression analysis for samples of the subset to identify genes differentially expressed implicated in the weak correlation of the subset.

13. The method of claim 12, further comprising identifying the biological process based on the identified genes.

14. The method of claim 13, wherein the identified biological process and identified genes include a pair selected from the group consisting of: proliferation and FLT1; tumor invasion and COL4A2; immune response and STAT1; angiogenesis and FGF18; and apoptosis and BBC3.

15. The method of claim 1, wherein the samples comprise whole slide images (WSIs), and the AI system divides each WSI into tiles, scores the tiles, identifies one of the highest- and lowest-scoring tiles for each slide, and calibrates scores for the remaining tiles based on the identified one of the highest- and lowest-scoring tiles for that slide.

16. The method of claim 1, further comprising, after the training step, using the AI system to identify a risk of cancer for a patient from at least one test image of tissue from the patient.

17. The method of claim 16, wherein the cancer is breast cancer.

18. A pathology system comprising: providing a computer system comprising at least one processer coupled to memory having an AI system resident therein wherein the system is operable to output predicted disease risks for subjects from samples provided as inputs to the system; correlate the predicted disease risks to calculated risk scores from biomarker data from the subjects; select a subset of the samples having weak correlation between the predicted disease risks and the calculated risk scores; identify a biological process that is differentially active in the subset compared to all of the samples; and receive to the AI system additional samples known to represent the biological process.

19. The system of claim 18, wherein the samples comprises images of stained tissue slices.

20. The system of claim 18, wherein the samples comprise whole slide images of stained tissue sections.

21. The system of claim 18, wherein the AI system comprises a deep neural network.

22. The system of claim 21, wherein the deep neural network is trained by multiple instance learning from training data comprising (i) whole slide images (WSIs) of slides, where each slide is presented as a bag comprising multiple tiles of pixels from that slide, and wherein each slide; and (ii) a diagnostic outcome for each slide.

23. The system of claim 22, wherein the deep neural network selects informative tiles from each slide and passes features from the informative tiles for each slide to a neural network that aggregates the informative tiles and predicts a final slide-level classification for that slide.

24. The system of claim 18, wherein the biomarker data comprises gene expression data.

25. The system of claim 24, wherein the gene expression data is obtained by microarray or RNA sequencing.

26. The system of claim 18, wherein the selecting step comprises selecting the subset as being those samples sharing a defined range of the predicted disease risks or the calculated risk scores.

27. The system of claim 18, wherein the selecting step further comprises performing differential expression analysis for samples of the subset to identify genes differentially expressed implicated in the weak correlation of the subset.

28. The system of claim 27, further comprising identifying the biological process based on the identified genes.

29. The system of claim 27, wherein the identified biological process and identified genes include a pair selected from the group consisting of: proliferation and FLT1; tumor invasion and COL4A2; immune response and STAT1; angiogenesis and FGF18; and apoptosis and BBC3.

30. The system of claim 18, wherein the samples comprise whole slide images (WSIs), and the AI system divides each WSI into tiles, scores the tiles, identifies one of the highest- and lowest-scoring tiles for each slide, and calibrates scores for the remaining tiles based on the identified one of the highest- and lowest-scoring tiles for that slide.

31. The system of claim 18, further comprising, after the training step, using the AI system to identify a risk of cancer for a patient from at least one test image of tissue from the patient.
